(19) United States
(12) Patent Application Publication
(10) Pub. No.: US 2018/0247554 A1
(43) Pub. Date: Aug. 30, 2018

(54) SYSTEM AND METHOD FOR TREATMENT OF INDIVIDUALS ON THE AUTISM SPECTRUM BY USING INTERACTIVE MULTIMEDIA

(71) Applicant: SPEECH KINGDOM LLC, Los Angeles, CA (US)

(72) Inventors: David Kalmick, Los Angeles, CA (US); Lois Kalmick, Los Angeles, CA (US)

(21) Appl. No.: 15/907,256

(22) Filed: Feb. 27, 2018

Related U.S. Application Data
(60) Provisional application No. 62/464,278, filed on Feb. 27, 2017.

(51) Int. Cl. G09B 7/06; G16H 20/70

(52) U.S. Cl. CPC G09B 7/06; G16H 20/70

(57) ABSTRACT

A method of treating an individual on the autism spectrum by using multimedia is provided. The method includes: displaying, on a display panel, a first scene of a social story, the first scene comprising an avatar; playing, by a speaker, an audio file describing the first scene; displaying, on the display panel, a graphical video marker corresponding to the avatar after the audio file has played; playing, on the speaker, a related audio prompt in response to a user selecting the avatar; and displaying, on the display panel, a question and a plurality of possible answers to the question, the question being related to the related audio prompt.

### CROSS-REFERENCE TO RELATED APPLICATION

This utility patent application claims priority to and the benefit of U.S. Provisional Application Ser. No. 62/464,278, filed Feb. 27, 2017 and titled “SYSTEM AND METHOD FOR INTERACTIVE COMMUNICATION,” the entire content of which is incorporated herein.

### BACKGROUND

### 1. Field

Aspects of example embodiments of the present invention relate to systems and method for treatment of individuals on the autism spectrum by using interactive multimedia.

### 2. Related Art

Autism, also referred to autism spectrum disorder (ASD), refers generally to a range of developmental challenges. ASD individuals generally exhibit challenges with social skills and verbal and non-verbal communication, repetitive behaviors, sensitivity to noise and light, and attention deficit disorder. The term “spectrum” is used as every individual with autism will exhibit different symptoms or combination of symptoms and will express the different symptoms to different degrees.

As individuals on the autism spectrum get older, their difficulties relating to others can be a significant detriment to their personal growth and independence. For example, people must relate to and interact with many others every day, from classmates and teachers to parents and friends, and individuals on the autism spectrum (referred to herein as “ASD individuals”) may struggle with each of these social interactions, leading them to become reclusive.

One major component of ASD individuals' social difficulties lies with their struggle with perspective-taking. Perspective-taking is a process by which an individual views or considers a situation from another's point-of-view. Perspective-taking is related to other theories and emotions, such as theory of mind (ToM) and empathy. ToM, of which perspective-taking may be considered a sub-theory, refers to an individual's ability to attribute mental states to oneself and to others, and to understand that others have different beliefs, thoughts, feelings, emotions, perspectives, and intentions. Individuals on the autism spectrum may struggle, to varying degrees, to understand others' thoughts, beliefs, feelings, emotions, intentions, perspectives, etc.

While neuro-typical children generally develop an ability to consider others' thoughts, feelings, etc., individuals on the autism spectrum struggle with perspective-taking and may not even recognize that others have feelings and thoughts different from their own. This difficulty or inability to consider others' thoughts, feelings, perspective, etc. can be significant hurdle to an ASD individual becoming self-supporting. This difficulty can also be a detriment to the individual's learning by, for example, causing difficulties in school and other behavioral issues resulting from frustration with how others act.

After an individual is diagnosed as being on the autism spectrum, one aspect of treatment includes helping the individual understand his or her deficiencies and difficulty with perspective-taking and helping him or her learn common verbal and non-verbal cues others make that are related to their thoughts, feelings, etc. Generally, perspective-taking may be taught by talking with an individual, most often a child, and explaining how others feel based on their actions, such as explaining that crying generally indicates that someone is upset. Another method of teaching perspective-taking is asking an individual how he or she would make someone else feel better if they are crying, for example. However, crying can indicate happiness or physical pain, in addition to sadness, or may come with laughter. Similar to crying, sarcasm can be difficult for ASD individuals to understand. Accordingly, teaching perspective-taking is very time consuming as many verbal and non-verbal cues must be demonstrated in different ways and in different environments and situations so that the ASD individual can gain experience with different verbal and non-verbal cues in different settings and with different underlying motivations and emotions.

However, individuals on the autism spectrum have difficulty with social interaction, to the point that they may not even be able to interact with others enough to understand if they are upset. Further, individuals on the autism spectrum often express attention deficient hyperactivity disorder (ADHD), making the extended social interactions necessary to learn perspective-taking very difficult. In addition, one-on-one therapy can be very expensive, meaning that families with a limited budget or insurance coverage may have to forgo the intensive therapy recommended to assist ASD individuals with reaching their full potential.

### SUMMARY

The present invention is directed toward various embodiments of systems and method for treatment of individuals on the autism spectrum by using interactive multimedia.

According to an embodiment of the present invention, a method of treating an individual on the autism spectrum by using multimedia includes: displaying, on a display panel, a first scene of a social story, the first scene comprising an avatar; playing, by a speaker, an audio file describing the first scene; displaying, on the display panel, a graphical video marker corresponding to the avatar after the audio file has played; playing, on the speaker, a related audio prompt in response to a user selecting the avatar; and displaying, on the display panel, a question and a plurality of possible answers to the question, the question being related to the related audio prompt.

The graphical video marker may be a colored halo around the avatar.

The colored halo may be orange or yellow.

The method may further include displaying, on the display panel, a second scene in response to the user selecting one of the possible answers. The second scene may include the avatar.

The first scene may include a plurality of avatars, and one of the avatars may mimic the user's physical characteristics.

The method may further include recording, in a memory, a user's selection of the possible answers.

The method may further include replaying, on the display panel, the user's selection of the possible answers.

According to another embodiment of the present invention, a method of treating an individual with a social communication delay or disorder by using a plurality of electronic devices is provided. Each of the electronic devices includes a processor and a memory connected to the processor, and the method includes: displaying, on a first one of the electronic devices, a first scene of a social story and playing, on the first one of the electronic devices, an audio file describing the first scene, the first scene including an avatar; recording an input to the first one of the electronic devices by a user; and transmitting the input to the first one of the electronic devices to a second one of the electronic devices via a network connection.

The method may further include displaying, on the first one of the electronic devices, a second scene of the social story in response to an input to the second one of the electronic devices.

The method may further include concurrently displaying, on the first and second ones of the electronic devices, the second scene of the social story.

The displays of the first and second ones of the electronic devices may display the same information.

The method may further include concurrently playing, on the first and second ones of the electronic devices, the audio file describing the first scene.

The method may further include displaying, on the second one of the electronic devices, a second scene of the social story in response to an input to the first one of the electronic devices.

According to another embodiment of the present invention, a system for treating an individual on the autism spectrum by using multimedia includes a plurality of devices in communication with each other. Each of the devices includes: a processor; a speaker connected to the processor; a display panel connected to the processor and configured to display an image; and an input device connected to the processor and configured to receive an input; and a memory connected to the processor. The memory stores instructions that, when executed by the processor, causes the processor to: display a first scene of a social story on the display panel, the first scene including an avatar; play an audio file on the speaker, the audio file describing the first scene; display a graphical video marker corresponding to the avatar, on the display panel, after the audio file has played; play a related audio prompt, on the speaker, in response to a user selecting the avatar; and display a question and a plurality of possible answers to the question on the display panel, the question being related to the related audio prompt.

The user selecting the avatar on one of the devices may cause the processor of the one of the devices to communicate with the processor of another one of the devices.

A second one of the devices may be configured to remotely control a first one of the devices.

The devices may each be connected to a remote server via the Internet.

The graphical video marker may be a colored halo around the one of the avatars.

The processor may be configured to detect an input corresponding to the graphical video marker.

The memory may be configured to store inputs to the corresponding one of the devices.

This summary is provided to introduce a selection of features and concepts of example embodiments of the present invention that are further described below in the detailed description. This summary is not intended to identify key or essential features of the claimed subject matter nor is it intended to be used in limiting the scope of the claimed subject matter. One or more of the described features according to one or more example embodiments may be combined with one or more other described features according to one or more example embodiments to provide a workable method or device.

### BRIEF DESCRIPTION OF THE DRAWINGS

FIG. 1 is a flowchart illustrating a method of teaching perspective-taking according to an example embodiment;

FIG. 2 is a schematic drawing of a network configuration of a program for teaching perspective-taking to individuals on the autism spectrum according to an example embodiment;

FIGS. 3-14 are screenshots of a social story of a program according to an embodiment of the present invention;

FIG. 15 is a screenshot of a fundamental story of the program according to an embodiment of the present invention;

FIG. 16 is a screenshot of various games available to users on the program according to an embodiment of the present invention; and

FIG. 17 is a screenshot of a game of the program according to an embodiment of the present invention.

### DETAILED DESCRIPTION

The present invention is directed toward various embodiments of systems and method for treatment of individuals on the autism spectrum by using interactive multimedia.

According to embodiments of the present invention, an electronic device or devices are used to teach social skills, such as perspective-taking, to individuals on the autism spectrum (“ASD individuals”). The present invention is not limited to teaching ASD individuals. Embodiments of the present invention may also be used to teach social skills and the like to children and adults with social communication delays and disorders. A program (e.g., a computer program or application) may run on the electronic device(s) to provide social stories or games to a user (e.g., a student). The user may be represented in the program by a cartoon-like avatar meant to imitate the user so that the user can more easily become immersed in the social story. The program may use multimedia, such as a combination of still and moving images, graphical indicators, called Graphical Video Markers (GVMs), audio, and text to retain the user's attention for an extended period of time. Each social story or game in the program may have one or more associated templates. The templates define how the story or game progresses and how the story or game is presented to the student and/or supervising adult, as further described below. The templates may also define which settings are changeable or adjustable by the user or supervising adult and which settings are locked (e.g., which settings are not changeable by the user or supervising adult). The locked settings may only be changed by updating the program, which may be accomplished via an over-the-air software update or the like.

It has been found that individuals on the autism spectrum are drawn to electronic devices, such as computers, tablet computers, smart phones, etc., and are able to concentrate on these devices for relatively long periods of time. Thus, while an ASD individual may quickly lose interest in social interactions, such as with a teacher, therapist, or parent, the same ASD individual is often able to concentrate on an electronic device for a much longer period of time. Further, one key aspect of teaching social skills to ASD individuals, such as perspective-taking, is repetition. Thus, electronic devices are especially suited to teaching social skills to ASD individuals.

To more accurately represent the physical world, a program (e.g., a software program or application), according to embodiments of the present invention, running on an electronic device, such as a tablet computer or smart phone, may display a cartoonish reproduction of a real-world environment with which the ASD individual may be familiar. For example, the program may display a classroom environment (e.g., a cartoonish reproduction of a classroom environment), a home environment (e.g., a cartoonish reproduction of a classroom environment), etc. By displaying the user's avatar in an environment with which the ASD individual is familiar, the user is more likely to retain the lessons taught by the program, described further below, because the lessons more easily translate into real-world interactions between the ASD individual and others. That is, the user can think back to certain social stories he or she played on the program when the user faces a real-world situation and determine how best to react based on lessons learned from the program.

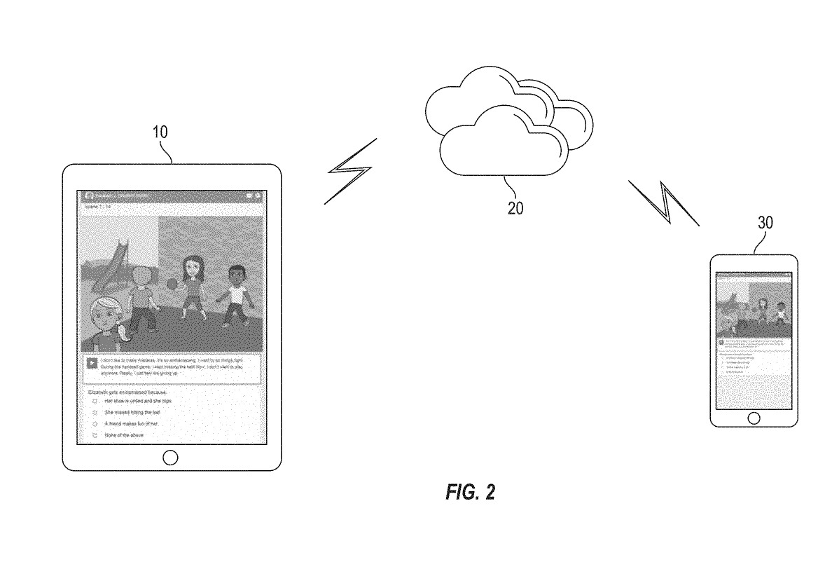

The program may be configured to run on a device or devices including a display panel and a touch screen panel, such as a smart phone, tablet computer, etc. Such devices generally include a processor, memory connected to the processor, a speaker configured to output sound, a display panel configured to display an image, and a touch screen panel arranged over the display panel and configured to receive a touch input. However, the present invention is not limited to devices using touch screen panels as input devices. The program may also be configured to run on devices including computer mice or touch pads as input devices, such as desktop and laptop computers. Further, the program, according to some embodiments, may be configured to run on various suitable operating systems, including Microsoft Windows, MacOS, Apple iOS, Android, etc. In some embodiments, the program may run entirely on a single device, and in other embodiments, the program may be concurrently shared among a plurality of different devices. For example, the program may run on a remote server (e.g., a cloud-based server) 20 (see, e.g., FIG. 2), and various different devices (e.g., various client devices) 10, 30 may be connected to the remote server 20 such that a single session may be shared between the client devices 10, 30.

Further, the program according to embodiments of the present invention may display cartoonish representations (e.g., avatars) of certain individuals with whom the ASD individual may interact in addition to the user's own avatar. For example, in the classroom setting, a cartoonish representation of a teacher and/or other students of similar age to the ASD individual may be displayed. In the home environment, an adult and/or other children may be displayed. By displaying cartoonish representations of environments and individuals that are familiar to the ASD individual, the social skills taught by the program are more likely to be applied by the ASD individual in the real world.

According to embodiments of the present invention, a series of Graphical Video Markers (GVMs) paired with Related Audio Prompts (RAPs) are utilized to teach ASD individuals various social skills, such as perspective-taking, and help ASD individuals better understand how others process and/or react to certain situations. As will be described further below, the GVMs may include, for example, yellow or orange halos around avatars; however, the present invention is not limited thereto.

ASD individuals are particularly perceptive of audio and visual prompts and cues, so the combination of GVMs and RAPs provides ASD individuals with stimulation to continue using the program for relatively long periods of time. For example, the ASD individuals may not even realize they are improving their social skills and may think they are merely playing a game.

To this end, the program, according to example embodiments, provides social lessons (or social stories) that employ GVMs to indicate that a certain individual, represented by an avatar in the program, has a thought and/or is experiencing an emotion. A single social lesson may be referred to as a “session.” During a session, a story may be told (e.g., a story audio file may be played) describing the displayed scene or some background information about the displayed scene. The story audio may correspond to displayed textual overlay on the screen so the user can also read the story. The scene may be a still image, a video clip, or a combination thereof. After the story has played, a GVM, such as a yellow halo, appears around the avatar having a thought or emotion. When the user selects (e.g., clicks on) the avatar with the GVM, a RAP, such as a pre-recorded or generated sound file, plays. The sound files, such as the story audio and the RAP, may be pre-recorded voice overs, computer generated from a stored text file, or a combination thereof. The RAP may be a sentence or multiple sentences that describe how the character with the GVM is feeling or a thought, emotion, feeling, etc. the character is experiencing based on the displayed situation. However, the present invention is not limited to feelings, thoughts, and emotions. Other social stories focus on people and friendship skills, emotional and physical self-regulation, executive functioning and life skills, self-awareness, inattention, and impulsivity, and safety at home, school, and in the community. Some more advanced social stories may include questions relating to different topics, further challenging users.

In some cases, multiple RAPs may be played, describing how different characters in the scene are feeling about the situation displayed and described by the story audio. The RAP may be associated with a visual text overlay. In some embodiments, the RAP may be replaced by the visual text overlay, for example, when the device is muted or if the ASD individual is sensitive to sound.

After listening to and/or reading the RAP, the user is presented with a question and number of possible answers. The question may relate to the emotional state of the avatar associated with the GVM and RAP. The question may force the user to consider the displayed situation from the avatar's position, thereby improving their social skills. For example, the RAP may be related to a certain action or interaction between the displayed avatars. However, the GVM may appear around one of the avatars not specifically involved with the action or interaction described by the RAP. Thus, the user must consider the situation (e.g., the action or interaction) from the designated avatar's perspective, regardless of the user's own perspective (e.g., the perspective of the user's avatar as displayed on the screen). The possible answers to the question may include feelings such as happy, sad, frustrated, excited, proud, embarrassed, etc. In some cases, the options may include words that are not feelings or emotions to further test the user as to his or her understanding of feelings or emotions more generally.

When the user selects the correct feeling or emotion (e.g., the correct answer), the social lesson continues to a next scene. When the user selects the incorrect feeling or emotion, the program may alert the user that an incorrect answer was selected and may either allow the user to select another feeling or emotion or may proceed to the next scene without having the user select the correct answer.

The various scenes of the social lesson may replicate a typical set of interactions experienced by the user in the real world. For example, one social lesson may involve a typical school day, starting at arriving at school and proceeding through various interactions with classmates, teachers, a therapist or counselor, etc.

In some embodiments, the user (or a supervising adult, such as a parent, teacher, therapist, etc.) may design an avatar to be displayed in the program. For example, the user may select an avatar's gender, hair style and color, skin tone, age, attire, etc. It is intended that the user will generate an avatar that reflect his or her own appearance or perceived appearance or that the supervising adult will design an avatar that reflects the user's appearance. In some embodiments, the user (or supervising adult) may also design other avatars, reflecting individuals the user may interact with in the real world. For example, the user may design avatars reflecting parents, teachers, a therapist, friends, classmates, etc.

ASD individuals often struggle with facial recognition. This difficulty can manifest in different ways, including difficulty recognizing faces altogether or difficulty recognizing certain facial features. In both instances, an ASD individual may struggle to perceive important facial clues, such as pursed lips, arched eyebrows, etc., that provide insights as to how a person is feeling or a person's emotional state. By providing the customizable avatars, the user can not only place him or herself “into” a particular situation, they can pseudo-interact with their parents, teachers, therapists, etc. and learn to recognize facial cues, easing the transition from skills learned in the program to real-world situations. Further, the user can improve their facial recognition skills by interacting with cartoonish representations of people they interact with in the real world, thereby limiting the number of new “faces” the user must learn and helping the user learn the distinguishing characteristics of the faces he or she sees on a regular basis.

In some embodiments, the avatars may have changing or variable facial features. For example, when a social situation is presented in the program in which a classmate is frustrated or mad, that classmate's avatar may have pursed lips, a clenched jaw, narrowed eyebrows, etc. Thus, in addition to the RAP and/or textual overlay, the user may be able to connect certain facial characteristics with certain moods, feelings, or emotions. Because the avatars may be designed to reflect the user's real-world classmates, for example, the user is able to more easily take the facial recognition skills learned by using the program and apply them to real-world situations.

By continued, repetitive use of the program, an ASD individual begins to connect certain social situations or social cues with certain feelings, perspectives, etc. of others. Thus, ASD individuals can improve their social skills by improving their perspective-taking skills and may begin to subconsciously connect situations encountered in the real world to those experienced or practiced in the program.

Some embodiments of the present invention include recording and replay functionality. Hereinafter, reference will be made to the program storing information. The information may be stored in transitory or non-transitory memory, locally or remotely, as would be understood by those skilled in the relevant art.

For example, the program may store the user's selected feelings or emotions (e.g., the selected answers) during a session and may allow a supervising adult to replay the user's session. The program may, in some embodiments, play through the session, showing how the user answered each question, or may provide a summary of the questions and how the user answered each in summary form, such as a textual list. The program may also provide both review options and may allow the supervising adult to select how she wants to view the results.

The program may store the results of many sessions over time. For example, the user may complete one social lesson a plurality of times, and the program may store the results of each of the plays through the one social lesson. Thus, the supervising adult can see how the user has progressed and is progressing by completing the social lesson multiple times and can better determine which emotions or feelings the user is struggling to understand. Then, the supervising adult can select social lessons that target the specific emotions or feelings the user is struggling to understand.

Also, some embodiments include progress tracking. In addition to the recording and replay functionality, the program may be able track the progress of a number of users. For example, when the program is used by a plurality of users in a same class or by a same therapist, the teacher or therapist may track the users' progress through different social lessons. By tracking the users' progress, the teacher or therapist may be able to determine trends in the users' learning, such as particular emotions or feelings the users are able to readily determine and other emotions or feelings the users cannot readily determine. Thus, the teacher or therapist can tailor lesson or treatment plans with the users and can determine which social lessons the users should play to best improve the users' social skills. In some embodiments, the program may review the results of the users' various sessions and may provide the teacher or therapist with a list of emotions or feelings with which the users are comfortable. For example, the program may output a list of emotions or feelings ranked in order from most comfortable, which could mean they are most often selected correctly by the users, to least comfortable, which could mean they are least often selected correctly by the users.

As described above, in some embodiments, the program may run on a remote server 20 and on different client devices 10, 30 (see, e.g., FIG. 2). For example, the remote server 20 may run the program and the different devices (e.g., client devices) 10, 30 may run clients of the program. In such an embodiment, one of the devices 10 may be a “supervising adult” client and another device or other devices 30 may be a “student” client. That is, one device 10 may be running the supervising adult client and the other device(s) 30 may be running a student client.

Different sessions conducted by different devices or device pairs may be expressed as a plurality of database entries in the remote server 20. For example, a UserStory column in the database indicates the different social stories that are available to be played by the student clients. When a new story is created and added to the remote server 20, a new UserStory entry is created in the database, and the devices 10, 30 will receive an update that a new story is available to be played. The remote server 20 may also include information relating to individual sessions running on the different clients 10, 30 and may store information about each session, including what social story is being run, what scene each session is on, a unique deviceID for the particular device running the program, which user is playing, etc.

As discussed further below, the supervising adult client 10 may control certain aspects of the student client(s) 30 to monitor, track, and control the student's experience using the program. The supervising adult client 10 and the student client 30 may be connected to each other by, for example, a wide area network (WAN), such as the Internet, or a local area network (LAN).

In a typical situation, a supervising adult (e.g., a parent, therapist, or teacher) may use the device running the supervising adult client 10 and an ASD individual may use the student client 30. In a classroom setting or the like, a plurality of ASD individuals may use different student clients 30. The supervising adult client 10 may be able to monitor and/or control each of the different student clients 30. For example, the supervising adult using the supervising adult client 10 may be able to switch between monitoring (e.g., may be able to “drop in”) the different student clients 30. The supervising adult may receive a notification when one of the students selects an incorrect emotion or feeling in a session or when one of the students remains on a certain scene for an extended period of time. One characteristic of some ASD individuals is the tendency to perseverate. In the context of this program, perseveration may be characterized by an ASD individual repeatedly selecting a GVM to hear a RAP without selecting the perceived emotion or feeling (e.g., without answering the question). When the supervising adult is alerted to this behavior on the supervising adult client 10, the supervising adult may drop into the user's session on the student claim 30 and may advance the student's session to the next scene to break the student's cycle.

That is, the supervising adult, using the supervising adult client 10, may be able to control the student client 30 by, for example, advancing to the next scene or refreshing the display on the ASD individual's device 30 to regain the user's attention. By allowing remote control of the student clients 30 by the supervising adult client 10, one teacher, therapist, parent, etc. may monitor progress of a number of users and ensure they are completing the sessions without breaking the users' focus or concentration on the program.

The program provides a number of options or settings which are customizable for different users along with a number of options or settings which are locked. Taken together, these different options (or settings) are called a “template.” The programmer determines which settings of the templates are visible to the user or supervising adult (e.g., able to modified by the user or supervising adult) and which settings are not visible. The programmer also determines the initial settings for the template. For example, the programmer may determine that color should be removed from the new story to prevent the users from simply making a color association rather than understanding the emotions or feelings behind the story. The programmer may further determine that the color setting should not be visible to the user or supervising adult as changing this setting would have a detrimental impact on the story's ability to teach the user a lesson. The template may be stored remotely (e.g., on a remote server) and/or may be downloaded and stored locally on a device.

When the program is started on a device, an opening screen may present the user with different program modes, such as fundamental stories, social stories, and games. When the user selects a certain mode, the user is presented with particular templates (e.g., different stories or games). When the user selects a certain template, that template may be loaded onto the device, either from a local memory or from a remote server (e.g., the could) and a new session is started.

Next, the user or supervising adult is presented with various options and settings. The displayed settings and options are the settings and options that the template indicates are visible. The visible settings and options are the settings and options that may be modified by the user or supervising adult. Examples of visible settings include, but are not limited to, whether or not the user is a student or supervising adult (e.g., whether or not the story or game should run in student mode), whether or not the user can advance forward and/or backwards between scenes, whether or not the narrative text is displayed, whether or not the avatar's thoughts are displayed as text, the maximum number of times a sound file can be played on a single scene, audio volume, audio speed, and whether or not to indicate correct answers.

As another example, game mode templates may include, as some examples, the following visible options and settings: whether the game is to be run in student mode, an option to manually select the picture sets to be presented during the game, a number of sets per round, a number of choices per set, maximum number of attempts allowed per set, automatic advance (e.g., automatic advance to the next set after the maximum number of attempts is met), options to prompt the user on correct and/or incorrect selections, and a maximum number of times an audio file can be played per set. When the supervising adult selects the option to manually select sets, a window appears displaying all of the stored sets. The supervising adult can then scroll through the available sets and select the sets for the student to play. When the supervising adult does not select the option to manually select sets, the program will randomly select sets from the database.

Each user's particular visible settings for each template may be saved, either locally or remotely, as part of the user's profile. Thus, after an initial setup period (e.g., after a first time a particular story or game is played), during which a user's individual visible settings are selected, the program may store the particular settings. As such, when the user starts a new session of a story or game he or she has played before, the user's particular visible settings for that story or game may be loaded automatically.

In some embodiments, the program may suggest certain settings and options (e.g., a profile) based on the functioning of a particular ASD individual. For example, some ASD individuals are particularly sensitive to sounds. The program may suggest a noise-sensitive profile which has, for example, the volume lowered or muted altogether and instead provides only textual and graphical information to communicate information to the user. Upon selection of the noise-sensitive profile, the supervising adult may be presented with the various visible settings to further customize the user's profile. In this way, the suggested profile provides a baseline to build from or customize based around the individual user's particular needs or sensitivities.

The fundamental stories mode of the program may be the most basic social stories of the program and are designed for lower-functioning ASD individuals. This mode focuses on the most basic feelings and emotions and has a simplified interface, which is more suitable for lower functioning ASD individuals. For example, the fundamental stories focus on situations in which avatars have something to say rather than how they feel or their perspective of a situation. Put another way, the fundamental stories focus on what others express or wish to express, rather than how others feel and, as such, do not teach perspective-taking or ToM. The goal of the fundamental stories is to teach lower-functioning ASD individual basic human expressions and corresponding situations so the ASD individuals can progress into the social stories and begin to learn perspective-taking. In addition, different from the other modes, the GVM in the fundamental stories mode is an orange glow around an avatar.

The social stories mode is another mode of the program and provides an ASD individual with situational learning to improve his or her perspective-taking skills. Similar to the fundamental stories, social stories provide the user with various relevant situations, for example, situations at school with teachers or classmates or situations at home, such as chores, and allows the user to work through the challenging aspects of such situations. Because the user has designed an avatar in his or her likeness, and other avatars in the likenesses of his or her parents, teacher, therapist, classmates, etc., the user can more readily identify with the situation by imagining him or herself interacting with parents, teacher, therapist, classmates, etc. Compared to the fundamental stories, the social stories involve more complex emotions and feelings involving more people. Some example social stories include the concepts of personal space, making mistakes, and others' feelings.

By proceeding from the fundamental stories through the social stories, and augmenting games and worksheets, an ASD individual or an individual with social communication delays and disorders may be able to continue to learn, grow, and improve his or her social skills by continuing to use the program over time. For example, the user incrementally improves his or her language (e.g., nouns, verbs, pronouns, prepositions, verbs, opposites, occupations, sentence creation, etc.), conversation (commenting, answering questions, asking questions, staying on topic, adding to the conversation, exiting the conversation, etc.), and social skills (e.g., identifying gestures, eye contact and proxemics, etc.) by playing more advanced fundamental and social stories in the program over time. That is, the social stories build on the skills taught by the fundamental stories, and the individual stories within the fundamental and social stories may be incrementally more advanced and more challenging, allowing for a smooth transition to more difficult topics and material.

In addition, the program may provide printable worksheets that reinforce the concepts taught in the fundamental and social stories. For example, the worksheets may be outlines of scenes from the stories of the program. Thus, the user may be able to further interact with the scenes, outside of an electronic device, to become more immersed in the scenes and, therefore, are more likely to learn the lessons that are being taught in the stories.

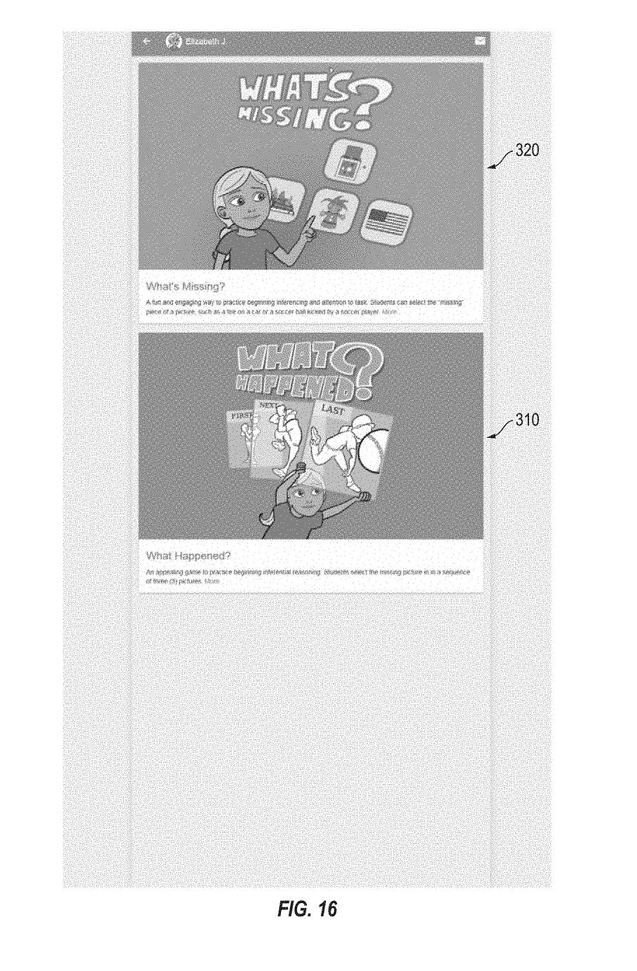

The games mode allows ASD individuals to improve their inferencing, inferential reasoning, and attention to task skills. Different from the fundamental stories and the social stories modes, the games do not tell a cohesive story but rather allow the user to work on particular skills in which ASD individuals are generally deficient. Some example games include the user selecting a missing piece of a picture, such as a tire on a car or a soccer ball kicked by a soccer player, to improve his or her inferencing and attention to task skills, or selecting a missing picture in a sequence of pictures to improve his or her inferential reasoning skills (see, e.g., FIG. 16). The games are customizable to various skill levels by, for example, limiting a number of possible answers or the position of the missing picture in the sequence of pictures, with a first picture omission being the most difficult and a last picture omission being the least difficult.

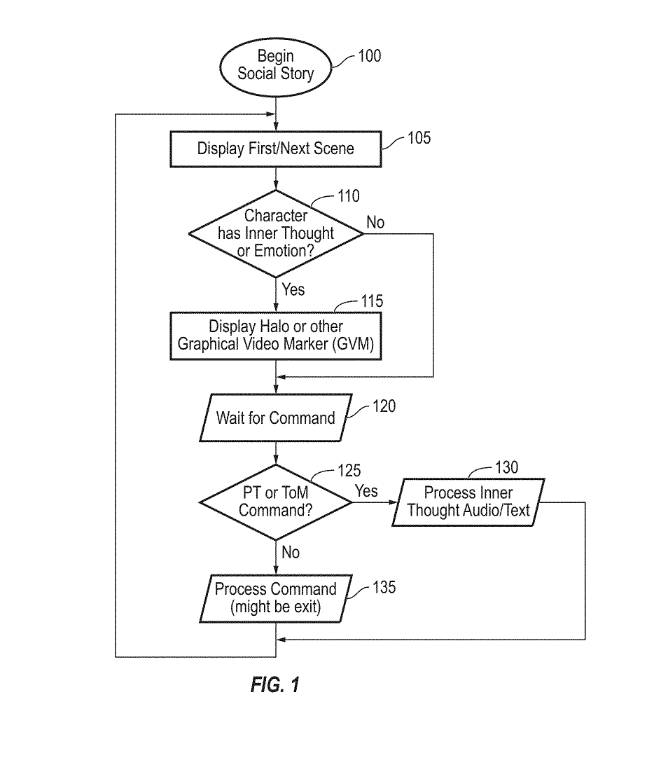

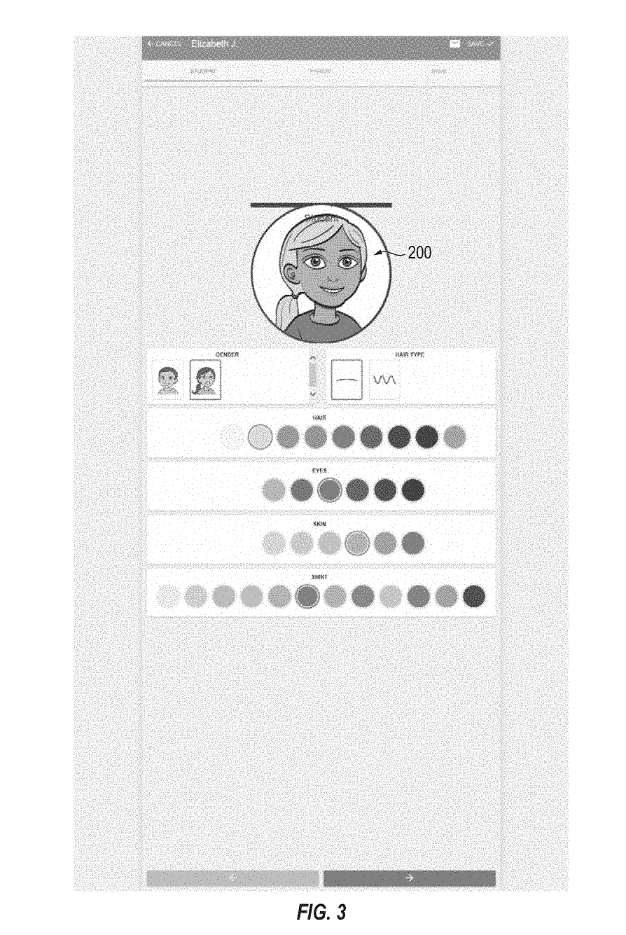

Referring to FIGS. 1 and 3-14, an example social story of the program according to an embodiment of the present invention will be described. Upon first starting the program, a user (e.g., a student) is prompted to design an avatar 200 in his or her own likeness (see, e.g., FIG. 3). Throughout this example, a female avatar 200 will be repeatedly referenced for ease of description.

Users are not required to design the avatar in his or her own likeliness and can design their avatar as they wish, based on how they perceive themselves or simply based on their personal desires. Further, the user or a supervising adult may design the avatar. For example, when a supervising adult is setting up the program for a student or child, the supervising adult may design the avatar to reflect the student or child's appearance. The user can select the avatar's gender, hair type, hair color, eye color, skin tone, and shirt color. The user's avatar 200 is saved either remotely or locally and may be a part of that user's profile. The program provides navigational buttons (e.g., left/right arrows) at the bottom of the screen, allowing the user to navigate between different screens.

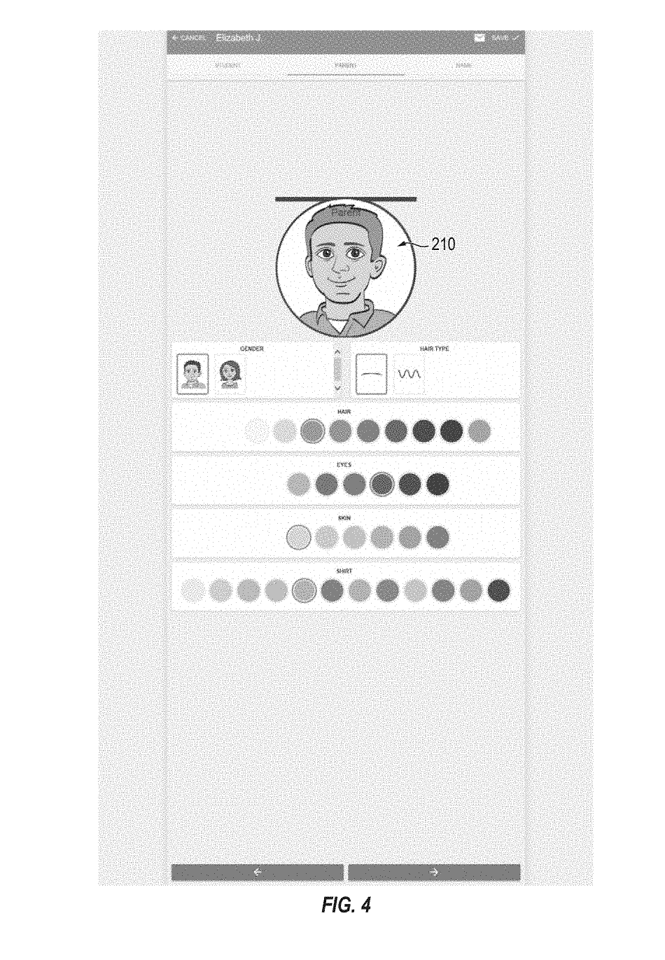

Next, the user is prompted to design an adult (e.g., a parent, therapist, teacher, etc.) avatar 210 (see, e.g., FIG. 4). Similar to designing the user's avatar 200, the user can select the adult avatar's gender, hair type, hair color, eye color, skin tone, and shirt color. The adult avatar 210 may be designed to mimic a parent, teacher, or therapist's likeness, although the user is not limited thereto. The adult avatar 210 is saved either locally or remotely as part of the user's profile.

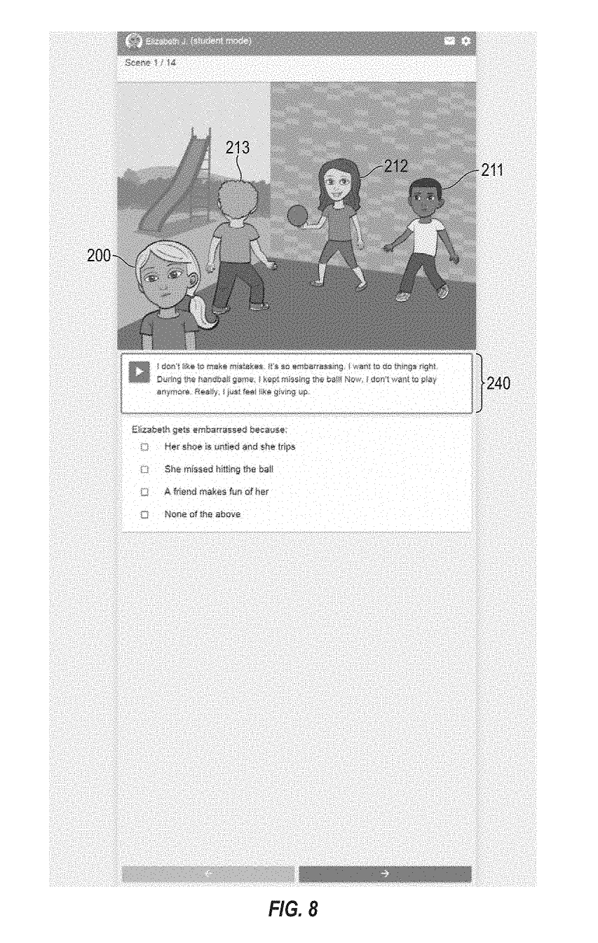

In some embodiments, additional avatars may be designed to mimic other parents, teachers, therapists, friends, classmates, etc. to improve the lifelike feel of the program to the user (see, e.g., avatars 211-213 in FIG. 8). Any additional avatars that are designed are also saved to the user's profile.

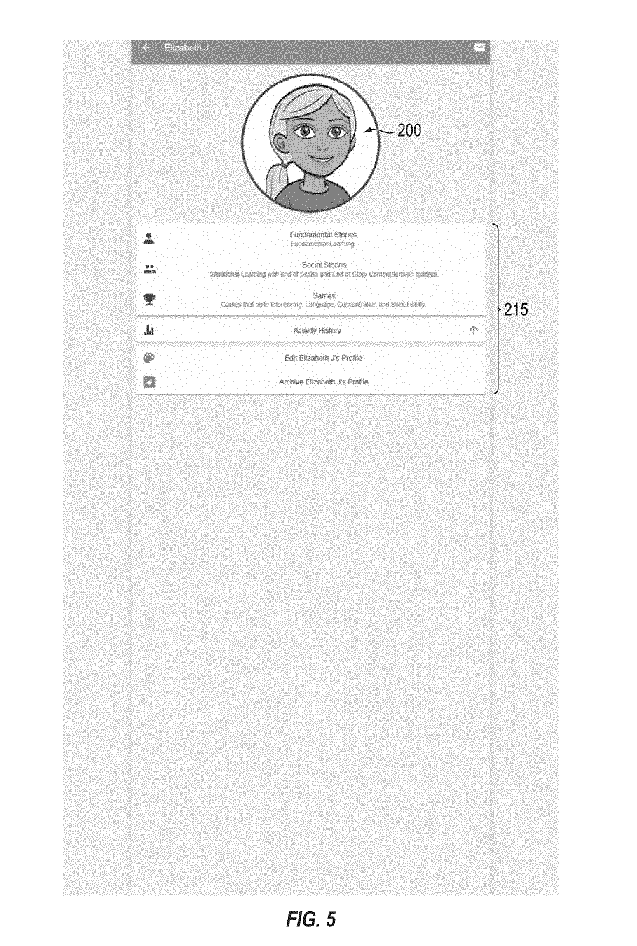

After the avatars are designed, the user reaches a home page (see, e.g., FIG. 5), which allows the user to select fundamental stories mode, social stories mode, games mode, history, and various settings 215. Upon future program start-ups, the user will be presented with the home page, rather than the avatar design pages, as the avatars are stored in the user's profile. If multiple users share a single device, the first screen displayed by the program display the different users' profiles that share the device, allowing the user to select his or her own profile. In this example, the user will select social stories. However, the user can also select a fundamental stories and games as other program modes, as discussed above.

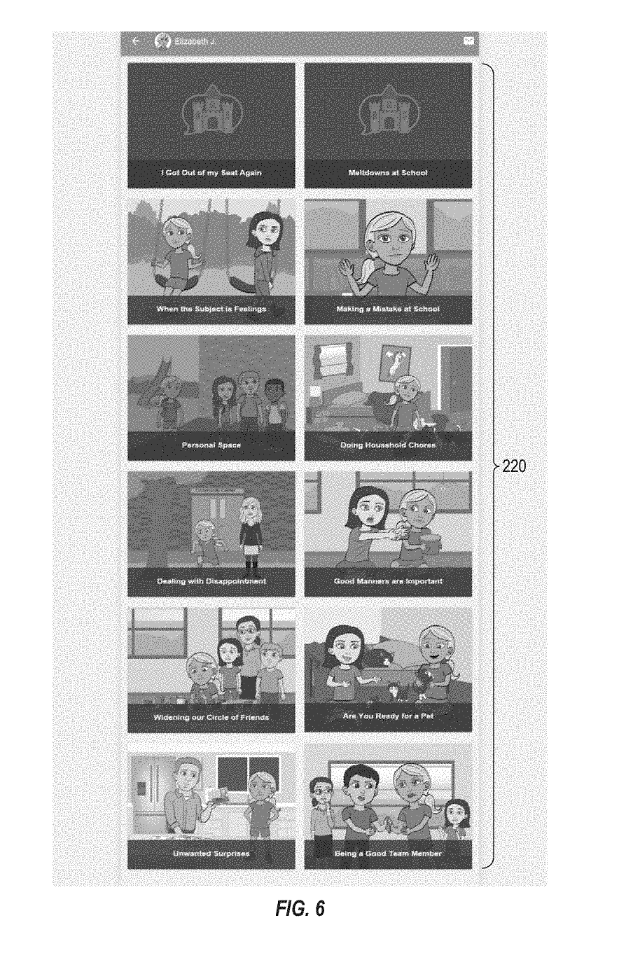

Referring to FIGS. 1 and 5-14, in this example, the user selects “Social Stories” from the homepage (see, e.g., FIG. 5). Next, the user is presented with various social stories 220 stored in the program (see, e.g., FIG. 6). As additional social stories are developed, they appear on this screen to be selected and played by the user. Here, the user selects which social story she would like to play.

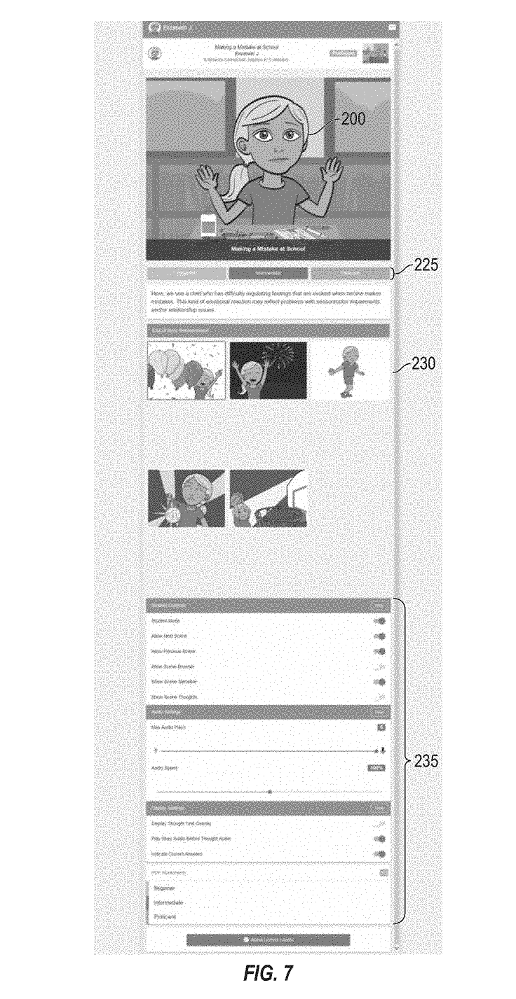

After selecting the social story she would like to play, the user selects the desired difficulty level 225, end of story reinforcement image or animation 230, and various visible settings 235 as defined by the template for that particular social story (see, e.g., FIG. 7). The difficulty level and settings 225, 235 may be pre-loaded from the user's profile but can also be changed on this screen before the session begins. In some embodiments, this screen may be skipped when the supervising adult does not want the user to have access to the difficulty level or settings. For example, one visible setting may be whether or not the user can change story-specific settings before beginning each story.

The end of story reinforcement animation 230 may be selected by the user or the supervising adult based on the user's particular preference. For example, the end of story reinforcement animation 230 may be considered a differential reinforcement response to the user completing the particular social story (or game or fundamental story in other instances). It has been found that ASD individuals in particular are receptive to certain animations involving their avatar. For example, one ASD individual may not be interested in seeing his or her avatar make a basket while another ASD individual may find the same animation very gratifying. Thus, by selecting a particular end of story reinforcement animation 230 that is motivating to the particular user, that user is further motivated to complete the social story (or game or fundamental story in other instances) and to complete additional social stories. Put another way, by accurately matching a particular end of story reinforcement animation 230 with a particular user, the user is more likely to continue using the program to improve his or her skills.

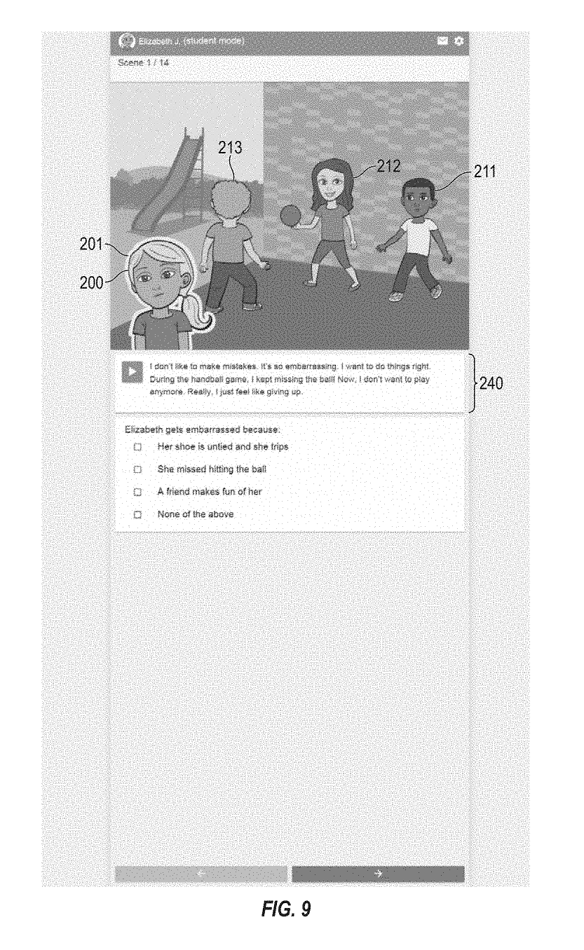

After the settings screen, or if the settings screen is bypassed, the program displays a first scene (105) (see, e.g., FIG. 8). A scene is a social situation that the user may find herself in, such as at school, home, etc. The scene displays the user's avatar 200, allowing the user to more easily visualize herself in the displayed situation, resulting in better skill improvement and retention. The scene may be a still image or a moving image (e.g., a movie or video clip). After a period of time, or when the user clicks anywhere on the image, a story audio file plays and the text corresponding to the story audio file 240 is displayed to the user. Some ASD individuals are exceedingly sensitive to sounds so, in that case, the user's profile may be set to not play the sound file. In this case, the text may highlight or bold at a normal reading speed so the user is inclined to read the text. The story audio file and associated text explains some background to the avatar's emotional state and/or the displayed situation. Upon completion of the sound file and/or the highlight/bolding of the associated text, and when the avatar is having a thought or emotion based on the presented story (110), a Graphical Video Marker (GVM) 201 appears on the screen (115). When the avatar is not having an inner thought or emotion, then no GVM appears. For example, the GVM 201 in the social stories mode may be a yellow halo that appears around the avatar having the thought or feeling (see, e.g., FIG. 9). In the fundamental stories mode, the GVM 201 may be an orange halo around the avatar.

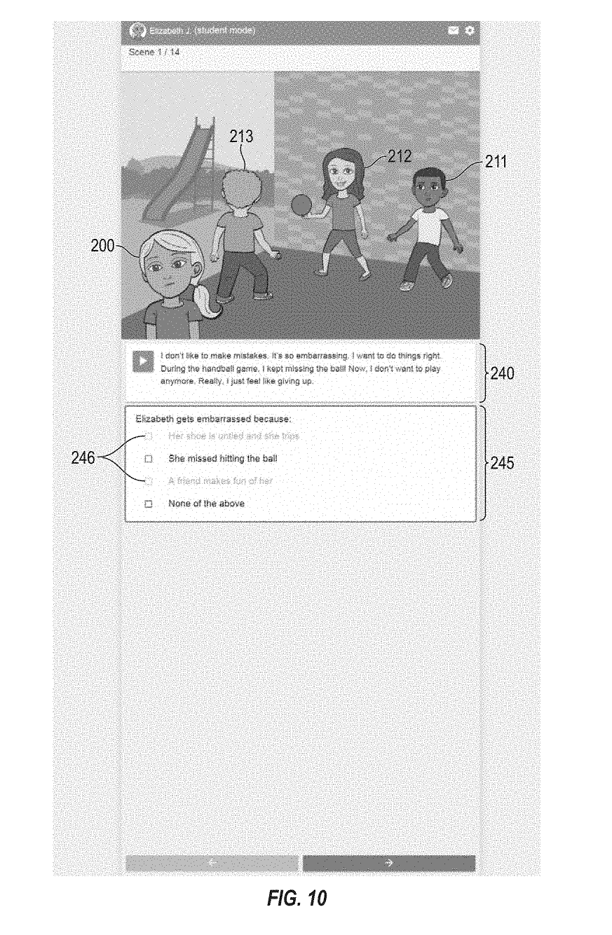

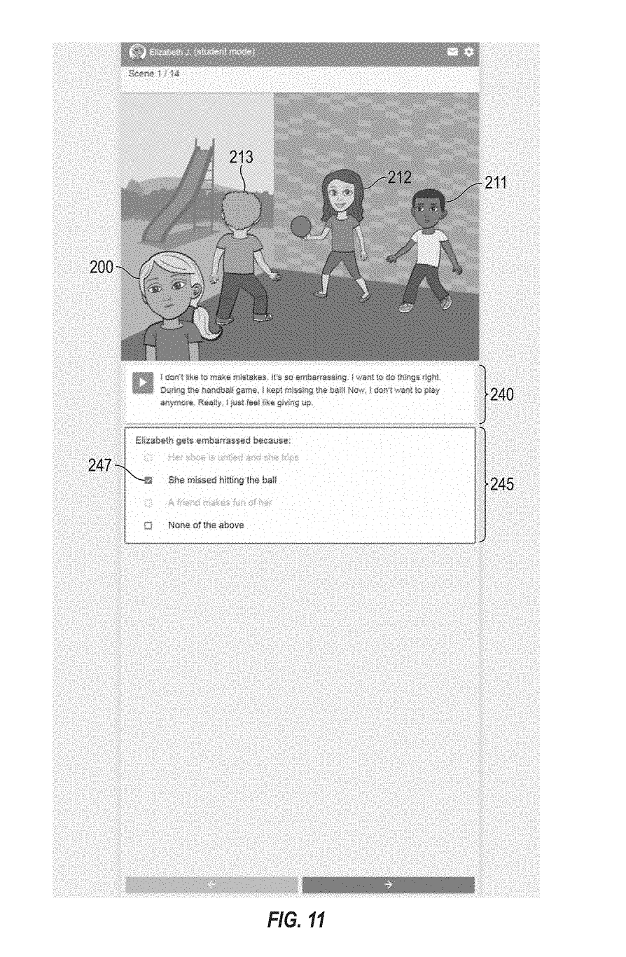

When the user selects (e.g., clicks on) the avatar with the GVM 200/201, a related audio prompt (RAP) plays. The RAP may further describe the emotional state of the related. After the RAP plays, a question appears below the scene and includes a question and multiple possible answers 245 (120) (see, e.g., FIG. 9). The question may relate to how the avatar associated with the GVM 200/201 is feeling or what an appropriate reaction would be for the avatar associated with the GVM 200/201 in the described situation. The number of possible answers varies based on the template, the selected difficulty setting, and/or as set at the beginning of the social story by the user or supervising adult, if such settings are visible. When the user makes an incorrect selection, that answer is greyed out and cannot be selected again 246 (see, e.g., FIG. 10). The user may be prompted to make another choice or to proceed to the next scene, depending on the settings (135). When the user makes the correct selection, a check box displays a check mark 247, indicating that the correct answer has been selected (125) (see, e.g., FIG. 11). After the correct answer is selected, another sound file may be play and/or text may be displayed, explaining why the answer is correct and/or furthering the story presented in the scene by explaining how the other avatars positively responded to the correct answer selected by the user (130).

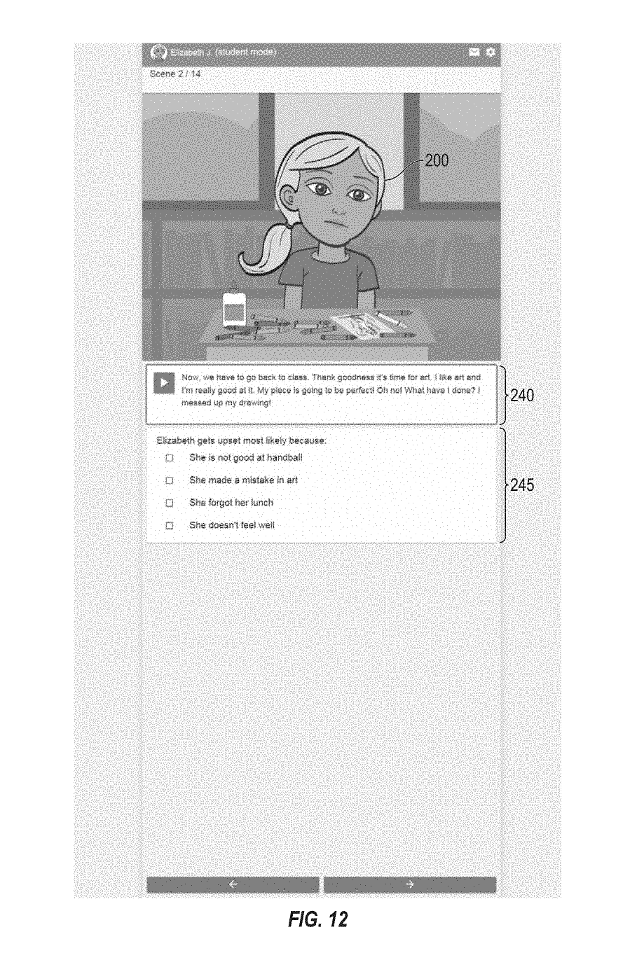

Then, a next scene is displayed (see, e.g., FIG. 12). For example, when the settings are set so that the user is not given an opportunity to correct an incorrect answer, the next scene may play immediately upon the incorrect selection of the prior scene. When the user is permitted, by the settings, to make multiple selections, the next scene may play upon selection of the correct answer in the prior scene and after any associated sound file is played and/or text is displayed.

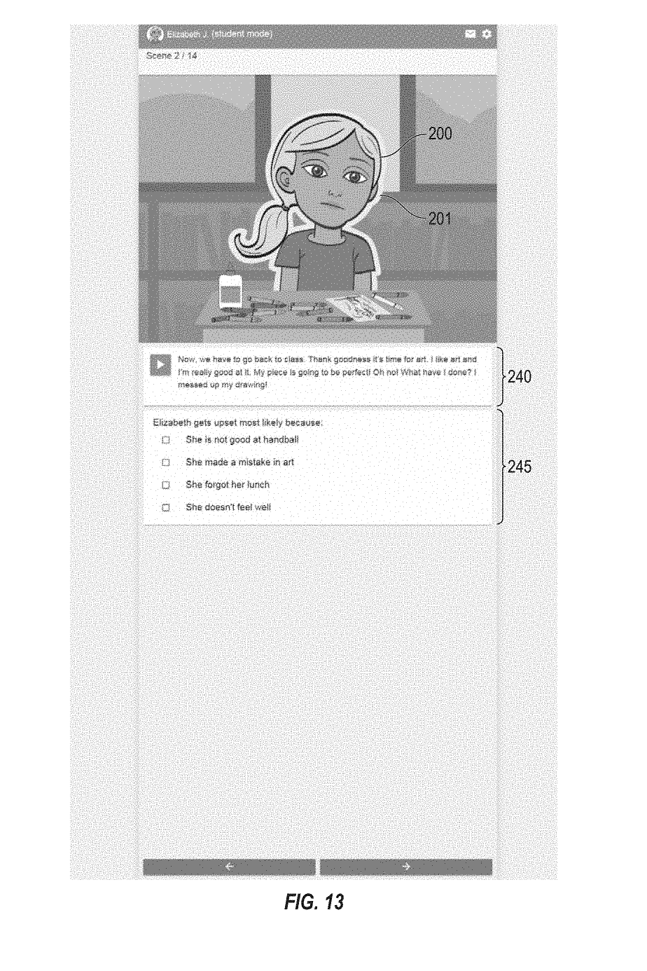

The next scene may further the avatar's story and may provide a different situation for the user to consider (see, e.g., FIG. 12). For example, when the social story is about a typical day at school for the user, the first scene may involve an incident on the playground and the second scene may involve a situation in a classroom. The second scene plays out substantially similarly to the first scene regarding the story audio file, the GVM, the RAP, selection of answers, and playing of related audio or display of text (see, e.g., FIG. 13).

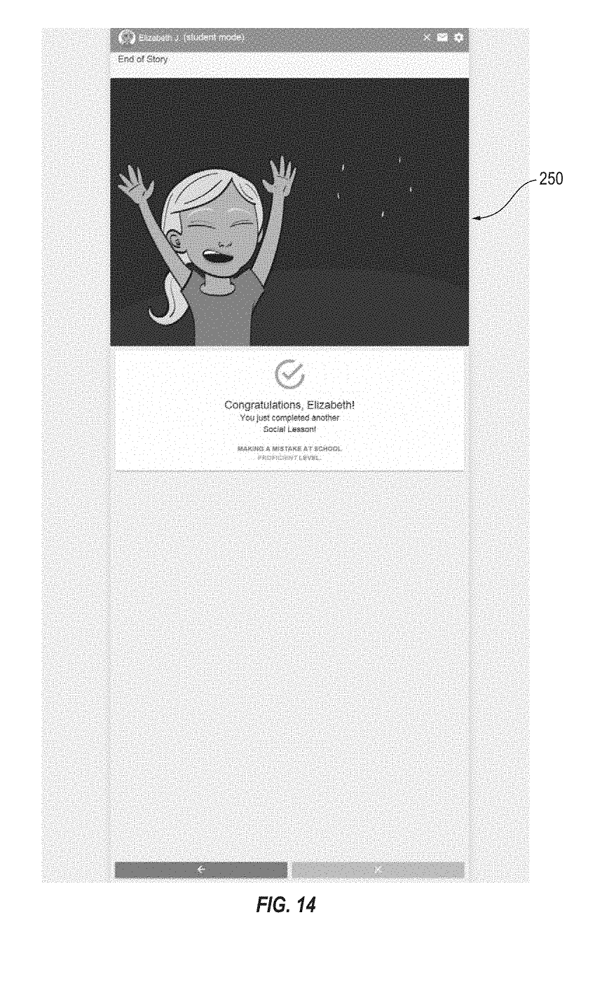

After completion of the final scene of a social story, an end of story reinforcement animation 250 is displayed congratulating the user on completion of the social story (see, e.g., FIG. 14). The end of story reinforcement animation 250 is animation that includes the user's avatar doing an action, such as dancing, moonwalking, watching fireworks, etc., reinforcing a positive overall experience for the user. As described above, the end of story reinforcement animation 250 may be a form of differential reinforcement. The results of the user's performance during the social story may be uploaded to the remote server 20 or saved on the local device for later review and/or replay by a supervising adult, as described above.

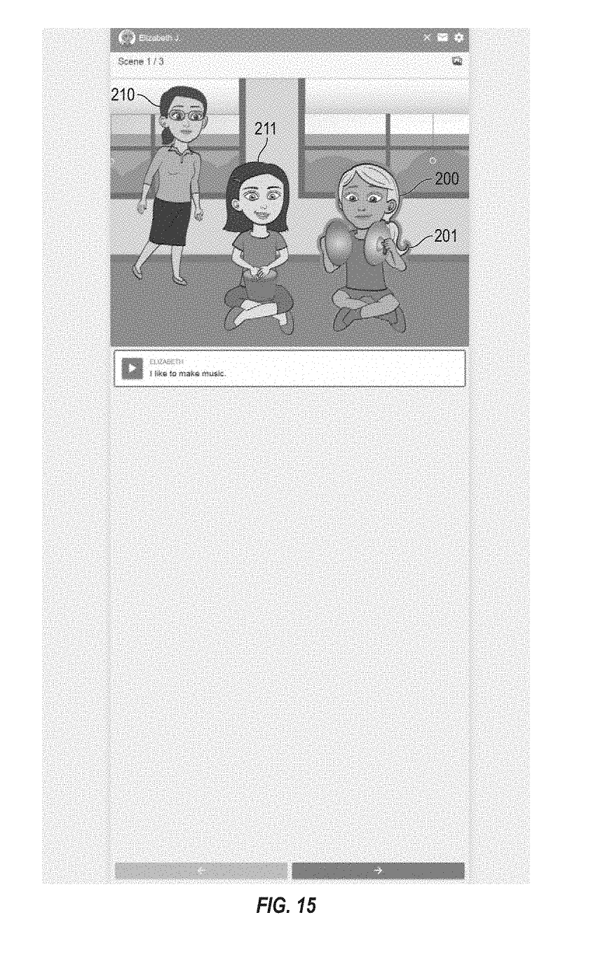

FIG. 15 shows an example screen of a fundamental story. As can be seen, in the fundamental story, much less information is presented to the user as the concepts to be taught in this mode are more basic. For example, the fundamental story shown in FIG. 15 teaches the user “parallel aware play,” or awareness of another child 211 playing nearby. Each scene in the fundamental story mode includes less text than in the social story mode to avoid overwhelming the lower-functioning ASD individuals that are intended to use the fundamental story mode. Further, instead of the GVM 201 being a yellow halo around an avatar, the GVM 201 in the fundamental story mode is an orange halo around an avatar.

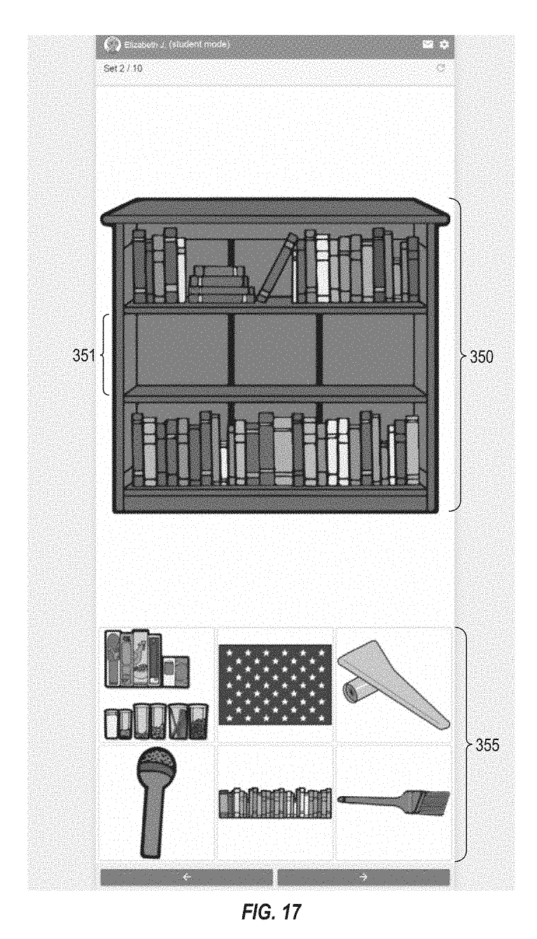

FIGS. 16 and 17 show example screens of the game mode. FIG. 16 shows two different games 310, 320 available to users in the game mode, both of which are described above. FIG. 17 shows a screen of the “What's Missing” game 320 shown in FIG. 16. In the “What's Missing” game 320, the user is presented with a scene (e.g., a still scene or moving scene) 350 that is missing some item 351. The user is presented with items 355 below the scene 350 that may complete the scene. The user then selects the missing item 351 from the presented items 355 and drags it into the scene 350. When the user “drops” the item into the scene, a sound file plays explaining if the user made a correct or incorrect selection. In some cases, when the user makes an incorrect selection, the sound file may explain, verbally, what is missing from the scene. Then the user can select what he or she has been told is missing from the scene verbally from the presented items. When the user makes the correct selection, the sound file may play explaining what was missing and is now not missing to ensure the user understands his or her selection. In FIG. 17, the scene is a bookshelf 350 with an empty shelf 351 and the presented items 355 include a microphone, paintbrush, a stack of books, etc. In this example, the correct missing item is the stack of books. Another example of the “What's Missing” game is a painter painting a fence but missing a paintbrush.

Example embodiments of the present invention have been described herein with reference to the accompanying drawings. The present invention, however, may be embodied in various different forms and should not be construed as being limited to only the embodiments illustrated herein. Rather, these embodiments are provided as examples so that this disclosure will be thorough and complete and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof may not be repeated.

It will be understood that, although the terms “first,” “second,” “third,” etc., may be used herein to describe various elements, components, and/or layers, these elements, components, and/or layers should not be limited by these terms. These terms are used to distinguish one element, component, or layer from another element, component, or layer. Thus, a first element, component, or layer described below could be termed a second element, component, or layer without departing from the spirit and scope of the present invention.

The terminology used herein is for the purpose of describing particular embodiments and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components but do not preclude the presence or addition of one or more other features, integers, steps, operations, elements, components, and/or groups thereof. That is, the processes, methods, and algorithms described herein are not limited to the operations indicated and may include additional operations or may omit some operations, and the order of the operations may vary according to some embodiments. As used herein, the term “and/or” includes any and all combinations of one or more of the associated listed items.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “example” is intended to refer to an example or illustration.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

A processor, such as a central processing unit (CPU), graphics processing unit (GPU), field-programmable gate array (FPGA), and/or any other relevant devices or components according to embodiments of the present invention described herein may be implemented utilizing any suitable hardware (e.g., an application-specific integrated circuit), firmware, software, and/or a suitable combination of software, firmware, and hardware. For example, the various components of the processor, CPU, GPU, and/or the FPGA may be formed on (or realized in) one integrated circuit (IC) chip or on separate IC chips. Further, the various components of the processor, CPU, GPU, and/or the FPGA may be implemented on a flexible printed circuit film, a tape carrier package (TCP), a printed circuit board (PCB), or formed on the same substrate as the processor, CPU, GPU, and/or the FPGA. Further, the described actions, operations, steps, acts, etc. may be processes or threads, running on one or more processors (e.g., one or more CPUs, GPUs, FPGAs, etc.), in one or more computing devices, executing computer program instructions and interacting with other system components to perform the various functionalities described herein. The computer program instructions may be stored in a memory, which may be implemented in a computing device using a standard memory device, such as, for example, a random access memory (RAM). The computer program instructions may also be stored in other non-transitory computer readable media such as, for example, a CD-ROM, flash drive, hard drive (HDD or SSD), or the like. Also, a person of skill in the art should recognize that the functionality of various computing devices may be combined or integrated into a single computing device or the functionality of a particular computing device may be distributed across one or more other computing devices without departing from the scope of the exemplary embodiments of the present invention.

Although the present invention has been described with reference to the example embodiments, those skilled in the art will recognize that various changes and modifications to the described embodiments may be performed, all without departing from the spirit and scope of the present invention. Furthermore, those skilled in the various arts will recognize that the present invention described herein will suggest solutions to other tasks and adaptations for other applications. It is the applicant's intention to cover by the claims herein, all such uses of the present invention, and those changes and modifications which could be made to the example embodiments of the present invention herein chosen for the purpose of disclosure, all without departing from the spirit and scope of the present invention. Thus, the example embodiments of the present invention should be considered in all respects as illustrative and not restrictive, with the spirit and scope of the present invention being indicated by the appended claims and their equivalents.

What is claimed is:

1. A method of treating an individual on the autism spectrum by using multimedia, the method comprising:
displaying, on a display panel, a first scene of a social story, the first scene comprising an avatar;
playing, by a speaker, an audio file describing the first scene;
displaying, on the display panel, a graphical video marker corresponding to the avatar after the audio file has played;
playing, on the speaker, a related audio prompt in response to a user selecting the avatar; and
displaying, on the display panel, a question and a plurality of possible answers to the question, the question being related to the related audio prompt.

2. The method of claim 1, wherein the graphical video marker is a colored halo around the avatar.

3. The method of claim 2, wherein the colored halo is orange or yellow.

4. The method of claim 1, further comprising displaying, on the display panel, a second scene in response to the user selecting one of the possible answers, the second scene comprising the avatar.

5. The method of claim 1, wherein the first scene comprises a plurality of avatars, one of the avatars mimicking the user's physical characteristics.

6. The method of claim 1, further comprising recording, in a memory, a user's selection of the possible answers.

7. The method of claim 6, further comprising replaying, on the display panel, the user's selection of the possible answers.

8. A method of treating an individual with a social communication delay or disorder by using a plurality of electronic devices, each of the electronic devices comprising a processor and a memory connected to the processor, the method comprising:
displaying, on a first one of the electronic devices, a first scene of a social story and playing, on the first one of the electronic devices, an audio file describing the first scene, the first scene comprising an avatar;
recording an input to the first one of the electronic devices by a user; and
transmitting the input to the first one of the electronic devices to a second one of the electronic devices via a network connection.

9. The method of claim 8, further comprising displaying, on the first one of the electronic devices, a second scene of the social story in response to an input to the second one of the electronic devices.

10. The method of claim 9, further comprising concurrently displaying, on the first and second ones of the electronic devices, the second scene of the social story.

11. The method of claim 10, wherein the displays of the first and second ones of the electronic devices display the same information.

12. The method of claim 9, further comprising concurrently playing, on the first and second ones of the electronic devices, the audio file describing the first scene.

13. The method of claim 12, further comprising displaying, on the second one of the electronic devices, a second scene of the social story in response to an input to the first one of the electronic devices.

14. A system for treating an individual on the autism spectrum by using multimedia, the system comprising a plurality of devices in communication with each other, each of the devices comprising:
a processor;
a speaker connected to the processor;
a display panel connected to the processor and configured to display an image; and
an input device connected to the processor and configured to receive an input; and
a memory connected to the processor, wherein the memory stores instructions that, when executed by the processor, causes the processor to:
display a first scene of a social story on the display panel, the first scene comprising an avatar;
play an audio file on the speaker, the audio file describing the first scene;
display a graphical video marker corresponding to the avatar, on the display panel, after the audio file has played;
play a related audio prompt, on the speaker, in response to a user selecting the avatar; and
display a question and a plurality of possible answers to the question on the display panel, the question being related to the related audio prompt.

15. The system of claim 14, wherein the user selecting the avatar on one of the devices causes the processor of the one of the devices to communicate with the processor of another one of the devices.

16. The system of claim 14, a second one of the devices is configured to remotely control a first one of the devices.

17. The system of claim 16, wherein the devices are each connected to a remote server via the Internet.

18. The system of claim 14, wherein the graphical video marker is a colored halo around the one of the avatars.

19. The system of claim 18, wherein the processor is configured to detect an input corresponding to the graphical video marker.

20. The system of claim 14, wherein the memory is configured to store inputs to the corresponding one of the devices.
